(19) United States
(12) Patent Application Publication
(10) Pub. No.: US 2008/0200724 A1
(43) Pub. Date: Aug. 21, 2008

(54) Process and Compound

(75) Inventors: Noel Hamil, Belfast (GB); Georges Hodges, Bedfordshire (GB); Ian Houson, Huddersfield (GB); Susan Pollard, Heckmondwike (GB)

Correspondence Address: KIRTON AND MCCONKIE, 60 EAST SOUTH TEMPLE,, SUITE 1800, SALT LAKE CITY, UT, 84111, US

(73) Assignee: NPIL Pharmaceuticals (UK) Ltd, Huddersfield (GB)

(21) Appl. No.: 11/917,090

(22) PCT Filed: Jun. 12, 2006
(86) PCT No.: PCT/GB06/02140

§ 371 Date: Jan. 9, 2008

(30) Foreign Application Priority Data
Jun. 10, 2005 (GB) 0511807.0

(51) Int. Cl. C07C 303/36

(52) U.S. Cl. 564/80

(57) ABSTRACT

The present invention relates to a process for preparing certain sulphonamide intermediates useful in the preparation of HIV inhibitors and to the crystal forms thereof.

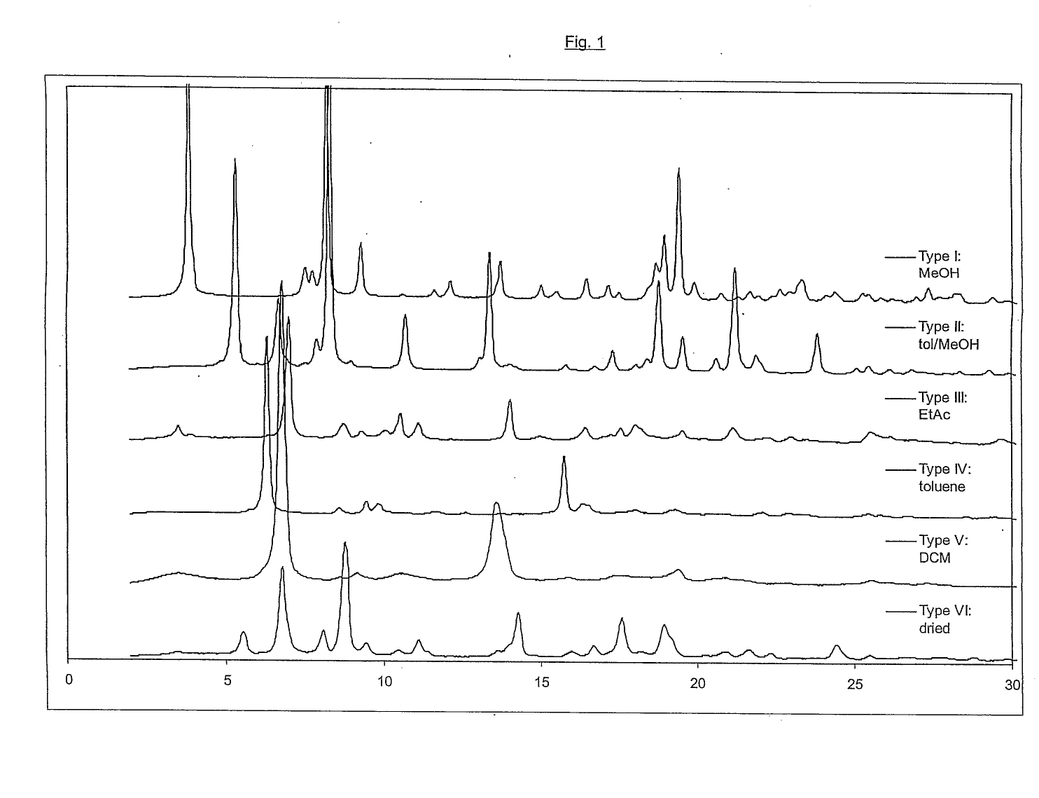

This invention relates to a process for preparing certain sulphonamide intermediates useful in the preparation of HIV inhibitors, and to the crystal forms thereof.

Human immunodeficiency virus (HIV), the causative agent of acquired immunodeficiency syndrome (AIDS), encodes three enzymes, including the well-characterized proteinase belonging to the aspartic proteinase family, the HIV protease. Inhibition of this enzyme has been regarded as a promising approach for treating AIDS. Hydroxyethylamine isosteres have been extensively utilized in the synthesis of potent and selective HIV protease inhibitors. However, this modern generation of HIV protease inhibitors has created an interesting challenge for the synthetic organic chemist. Advanced x-ray structural analysis has allowed for the design of molecules that fit closely into active sites on enzymes creating very effective drug molecules. Unfortunately, these molecules, designed by molecular shape, are often difficult to produce using conventional chemistry.

The modern generation of HIV inhibitors has structural similarities in a central three-carbon piece containing two chiral carbons that link two larger groups on each side (see, e.g., Parkes, et al, J. Org. Chem., 39:3656 (1994)). In general, the chemical bond from the central part to one of the larger groups is a carbon-nitrogen bond which is usually accomplished by reacting an epoxide with an amine. 2R,3S—N-isobutyl-N-(2-hydroxy-3-amino-4-phenylbutyl)-p-nitrobenzenesulfonylamide hydrochloride is a key intermediate in the synthesis of protease inhibitors (see, e.g., U.S. Pat. No. 5,585,397, U.S. Pat. No. 5,723,490 and U.S. Pat. No. 5,783,701 and WO94/0563). One process currently used to prepare 2R,3S—N-isobutyl-N-(2-hydroxy-3-amino-4-phenylbutyl)-p-nitrobenzenesulfonylamide hydrochloride is illustrated Scheme 1.

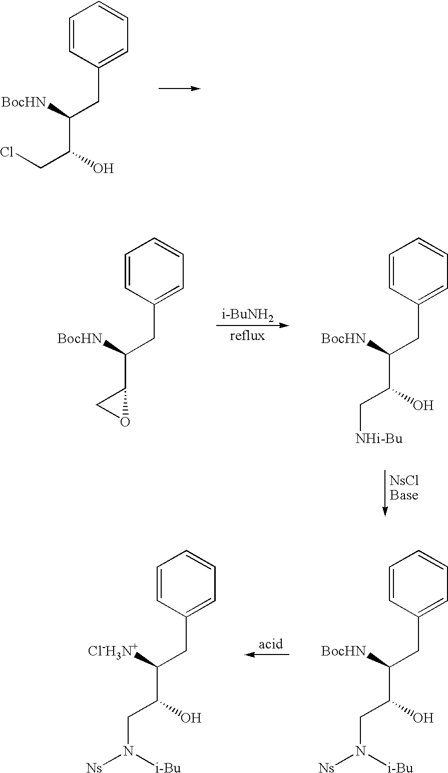

The 2R,3S—N-isobutyl-N-(2-hydroxy-3-amino-4-phenylbutyl)-p-nitrobenzene-sulfonylamide hydrochloride may be made in 4-step process starting from a commercially available (Aerojet Fine Chemicals (Sacramento, Calif.)) starting material, 2S,3S-chloromethylalcohol (2S,3S-CMA). This can be accomplished by first reacting the 2S,3S-CMA with sodium hydroxide in THF/ethanol to give the corresponding epoxide in 91% yield, and in second step, reacting the epoxide in toluene with excess isobutylamine at a temperature of 75° C. to 80° C. to give the diaminoalcohol. Reaction of diaminoalcohol with p-nitrobenzenesulfonyl chloride (i.e., nosyl chloride) in toluene at a temperature of 85° C. to 90° C., followed by removal of the Boc protecting group with aqueous HCl at a temperature of 85° C. to 90° C. gives 2R,3S—N-isobutyl-N-(2-hydroxy-3-amino-4-phenylbutyl)-p-nitrobenzenesulfonylamide hydrochloride, in 64% overall yield.

Alternatively, 2R,3S—N-isobutyl-N-(2-hydroxy-3-amino-4-phenylbutyl)-p-nitrobenzenesulfonylamide hydrochloride may be made according to an improved process described in U.S. Pat. No. 6,548,706 which avoids the need to prepare and isolate the epoxide. This improved process is claimed to result in higher yields of 2R,3S—N-isobutyl-N-(2-hydroxy-3-amino-4-phenylbutyl)-p-nitrobenzenesulfonylamide hydrochloride; while not sacrificing its purity. Thus, the 2S,3S-chloromethylalcohol (2S,3S-CMA) is reacted directly with isobutylamine, to give the diaminoalcohol. The diaminoalcohol is then reacted with nosyl chloride before removal of the Boc protecting group. The teachings of U.S. Pat. No. 6,548,706 are incorporated herein by reference.

However, in some cases it is preferable to isolate the BOC-protected 2R,3S—N-isobutyl-N-(2-hydroxy-3-amino-4-phenylbutyl)-p-nitrobenzene-sulfonylamide before further onwards reactions. The BOC-protected 2R,3S—N-isobutyl-N-(2-hydroxy-3-amino-4-phenylbutyl)-p-nitrobenzene-sulfonylamide is amenable to storage before further onwards reactions.

According to a first aspect of the present invention there is provided a process for preparing a BOC-protected 2R,3S—N-isobutyl-N-(2-hydroxy-3-amino-4-phenylbutyl)-p-nitrobenzene-sulfonylamide wherein the BOC-protected 2R,3S—N-isobutyl-N-(2-hydroxy-3-amino-4-phenylbutyl)-p-nitrobenzene-sulfonylamide is isolated from a solution comprising methanol and BOC-protected 2R,3S—N-isobutyl-N-(2-hydroxy-3-amino-4-phenylbutyl)-p-nitrobenzene-sulfonylamide.

Attempts to isolate BOC-protected 2R,3S—N-isobutyl-N-(2-hydroxy-3-amino-4-phenylbutyl)-p-nitrobenzene-sulfonylamide in the absence of methanol, for example using toluene or ethyl acetate as the sole solvent, gave very poor physical form material. This poor physical form material was fibrous in structure, forming extended mats similar to cotton wool, which retained solvent and rendered removal from reaction vessels arduous, even at low loading. Poor physical form material is therefore difficult to filter and can cause other handling difficulties.

The solution comprising methanol and BOC-protected 2R,3S—N-isobutyl-N-(2-hydroxy-3-amino-4-phenylbutyl)-p-nitrobenzene-sulfonylamide includes solutions wherein the BOC-protected 2R,3S—N-isobutyl-N-(2-hydroxy-3-amino-4-phenylbutyl)-p-nitrobenzene-sulfonylamide is fully dissolved in methanol or methanol containing solvent mixtures, and solutions wherein the BOC-protected 2R,3S—N-isobutyl-N-(2-hydroxy-3-amino-4-phenylbutyl)-p-nitrobenzene-sulfonylamide is partially dissolved in methanol or methanol containing solvent mixtures, for example slurries of BOC-protected 2R,3S—N-isobutyl-N-(2-hydroxy-3-amino-4-phenylbutyl)-p-nitrobenzene-sulfonylamide in methanol or methanol containing solvent mixtures.

The solution comprising methanol and BOC-protected 2R,3S—N-isobutyl-N-(2-hydroxy-3-amino-4-phenylbutyl)-p-nitrobenzene-sulfonylamide may be obtained by dissolving BOC-protected 2R,3S—N-isobutyl-N-(2-hydroxy-3-amino-4-phenylbutyl)-p-nitrobenzene-sulfonylamide in methanol or methanol containing solvent mixtures, or by slurrying BOC-protected 2R,3S—N-isobutyl-N-(2-hydroxy-3-amino-4-phenylbutyl)-p-nitrobenzene-sulfonylamide in methanol or methanol containing solvent mixtures. Alternatively, the solution comprising methanol and BOC-protected 2R,3S—N-isobutyl-N-(2-hydroxy-3-amino-4-phenylbutyl)-p-nitrobenzene-sulfonylamide may be obtained by direct addition of methanol to a solution comprising BOC-protected 2R,3S—N-isobutyl-N-(2-hydroxy-3-amino-4-phenylbutyl)-p-nitrobenzene-sulfonylamide or by solvent exchange, whereby the solution solvent is exchanged for methanol or a methanol containing solvent mixture, or by synthesing BOC-protected 2R,3S—N-isobutyl-N-(2-hydroxy-3-amino-4-phenylbutyl)-p-nitrobenzene-sulfonylamide in methanol or a methanol containing solvent mixture.

Methanol containing solvent mixtures include any solvent mixtures comprising methanol in which the BOC-protected 2R,3S—N-isobutyl-N-(2-hydroxy-3-amino-4-phenylbutyl)-p-nitrobenzene-sulfonylamide can be solubilised. Preferably, the solvent mixtures comprise organic solvents which are miscible with methanol. Preferred organic solvents include alkyl acetates, for example, methyl acetate, ethyl acetate, propyl acetate, for example isopropyl acetate, butyl actetate, for example isobutyl acetate or t-butyl acetate, and aromatic solvents, for example, toluene, benzene and xylene, and mixtures thereof.

Highly preferred methanol containing solvent mixtures include methanol/toluene mixtures, methanol/ethyl acetate mixtures and methanol/toluene/ethyl acetate mixtures.

When methanol containing solvent mixtures are employed it is preferred that the methanol content is greater than 10%, more preferably greater than 30% and most preferably greater than 40% by volume with respect to the total volume of solvents present.

When tertiary mixtures of methanol containing solvent mixtures are employed, the other non-methanol solvents are preferably present in ratios ranging from 1:99 to 99:1, more preferably in ratios ranging from 25:75 to 75:25, and most preferably in ratios ranging from 60:40 to 40:60, for example 50:50.

Preferably, the BOC-protected 2R,3S—N-isobutyl-N-(2-hydroxy-3-amino-4-phenylbutyl)-p-nitrobenzene-sulfonylamide is isolated by crystallisation or by precipitation techniques, for example by addition of an antisolvent.

In a preferred embodiment, crude BOC-protected 2R,3S—N-isobutyl-N-(2-hydroxy-3-amino-4-phenylbutyl)-p-nitrobenzene-sulfonylamide is dissolved by heating in methanol or a methanol comprising solvent mixture to form a saturated or partially saturated methanol containing solution. Optionally the saturated or partially saturated methanol containing solution is filtered to remove any insoluble material, then the saturated or partially saturated methanol containing solution is cooled to precipitate the BOC-protected 2R,3S—N-isobutyl-N-(2-hydroxy-3-amino-4-phenylbutyl)-p-nitrobenzene-sulfonylamide.

In another preferred embodiment, methanol is added to a heated saturated or partially saturated solution of BOC-protected 2R,3S—N-isobutyl-N-(2-hydroxy-3-amino-4-phenylbutyl)-p-nitrobenzene-sulfonylamide in a non-methanol solvent or solvent mixture, for example toluene, ethyl actetate or toluene/ethyl acetate mixture. Optionally the saturated or partially saturated methanol containing solution is filtered to remove any insoluble material, then the saturated or partially saturated methanol containing solution is cooled to precipitate the BOC-protected 2R,3S—N-isobutyl-N-(2-hydroxy-3-amino-4-phenylbutyl)-p-nitrobenzene-sulfonylamide.

The term saturated or partially saturated methanol containing solution refers to solutions at a reference temperature which comprise concentrations of dissolved solids, the concentration having an upper value defined by the maximum concentration achievable at the reference temperature and having a lower limit defined by the maximum concentration achievable at lower, end-point, temperature. Therefore, solutions having concentrations of dissolved solid between the upper and lower values at the reference temperature will result in precipitation of the dissolved solid when cooled to the lower, end-point temperature. The upper and lower concentration values are therefore dependent on the solubility characteristics of the material and the solvent mixture of choice. For solvent mixtures comprising methanol and one or more of toluene or ethyl acetate, typically the maximum solubility achievable at 60° C. is 24% w/w, and the lower solubility value, for an end-point temperature of 0° C., is typically 0.3% w/w. Therefore, for a crystallisation regime starting at 60° C. and ending at 0° C., a saturated or partially saturated solution in methanol and toluene, ethyl acetate admixtures, the concentration of BOC-protected 2R,3S—N-isobutyl-N-(2-hydroxy-3-amino-4-phenylbutyl)-p-nitrobenzene-sulfonylamide is in the range from 0.3 to 24% w/w.

When a saturated or partially saturated methanol containing solution is cooled to precipitate the BOC-protected 2R,3S—N-isobutyl-N-(2-hydroxy-3-amino-4-phenylbutyl)-p-nitrobenzene-sulfonylamide, it is preferred that saturated or partially saturated methanol containing solution is initially heated to over 40° C., more preferably to over 50° C. and most preferably to at least 65° C.

Cooling is preferably carried out slowly, whereby cooling is sufficiently slow to avoid secondary nucleation. Preferably cooling is carried out under ramping conditions as known in the art. More preferably, ramped cooling conditions start with a slow cooling rate and the cooling rate is gradually increased as time elapses. The cooling rate may be increased by a simple gradient increase or by multi-gradient increases, for example exponential increases.

In a preferred embodiment, a saturated or partially saturated methanol containing solution at an initial elevated temperature, preferably of from 45° C. to 75° C. is cooled stepwise, whereby the saturated or partially saturated methanol containing solution is cooled at a first cooling rate until a first temperature is reached, optionally the solution is held at this first temperature, preferably for 1 h or more, then cooling is recommenced at a second cooling rate until a second temperature is reached. Optionally the solution is held at this second temperature, preferably for 1 h or more, and optionally cooled further in one or more steps. Preferably the second cooling rate is slower than any subsequent cooling rates. When a final temperature is reached the BOC-protected 2R,3S—N-isobutyl-N-(2-hydroxy-3-amino-4-phenylbutyl)-p-nitrobenzene-sulfonylamide is isolated, preferably by filtration and optional washing of the isolated filtrate with solvent.

Preferably, the first cooling rate is less than 30° C./h and the first temperature is from 44° C. to 50° C. Preferably, the second cooling rate is less than 10° C./h, more preferably less than 4° C./h, and the second temperature is from 34° C. to 36° C. Subsequent cooling is preferably carried out at rates from 5° C./h to 30° C./h, more preferably between 5° C./h to 10° C./h. Preferably, the final temperature is from 0° C. to minus 10° C. The methanol containing solution is preferably held at 0° C. for up to 1 hr before recovering the crystalline BOC-protected 2R,3S—N-isobutyl-N-(2-hydroxy-3-amino-4-phenylbutyl)-p-nitrobenzene-sulfonylamide. Faster and slower cooling rates may be employed. While increased cooling rates may be accompanied by some deterioration of the physical form, slower cooling rates appear not to be detrimental to the physical form. In theory, there is no lower limit to the cooling rate, however slower cooling rates can result in accompanying increases in the time taken to carry out the crystallisation process, such time increases may impose some practical limits that make operation with extremely slow cooling rates over long periods of time less desirable at industrial scale.

Physical form can however be improved by ripening processes well known in the art. Preferred ripening processes include heating suspended BOC-protected 2R,3S—N-isobutyl-N-(2-hydroxy-3-amino-4-phenylbutyl)-p-nitrobenzene-sulfonylamide in methanol or a methanol containing solvent mixture at an elevated temperature, preferably from 25° C. to 45° C., for a period of time before slowly cooling to 0° C. The period of time is chosen to allow sufficient time for the physical form to change. Ripening processes may however prove time consuming, thus while the effects of over fast cooling may be ameliorated, additional time consuming steps may render the process less economical.

Preferably, as the saturated or partially saturated methanol containing solution is cooled, the saturated or partially saturated methanol containing solution is seeded with crystalline BOC-protected 2R,3S—N-isobutyl-N-(2-hydroxy-3-amino-4-phenylbutyl)-p-nitrobenzene-sulfonylamide. The quantity of seed crystal employed may be determined by methods known in the art.

The point of seeding can be derived from the solubility curve and by determining the point of turbidity change. When small quantities of seed are added while supersaturating the solution, the point of seeding is reached when turbidity increases on seeding. For example, from the solubility curve it is known that a 12.7% w/w solution will start supersaturating at 43.3° C. Seed crystals of the correct crystal form (Form II) were added incrementally from 44° C. while cooling. Turbidity increased at 42° C. indicating the point of seeding had been reached.

According to a further aspect of the present invention there is provide a crystalline BOC-protected 2R,3S—N-isobutyl-N-(2-hydroxy-3-amino-4-phenylbutyl)-p-nitrobenzene-sulfonylamide obtainable by the processes of the first aspect of the present invention.

We have found that in the crystalline state BOC-protected 2R,3S—N-isobutyl-N-(2-hydroxy-3-amino-4-phenylbutyl)-p-nitrobenzene-sulfonylamide is polymorphic. By using the processes of the present invention certain crystal forms can be obtained.

When methanol is used as the sole solvent, a crystalline BOC-protected 2R,3S—N-isobutyl-N-(2-hydroxy-3-amino-4-phenylbutyl)-p-nitrobenzene-sulfonylamide of Crystal Form I is obtained. The habit of Crystal Form I is prismatic and thus is amenable to easy filtration. When measured by X-ray diffraction (Siemens D5000 X-Ray Diffractometer, 20, CuKα radiation) Crystal Form I shows strong peaks at 3.9 and 8.2, moderate peaks at 9.3, 13.7 18.7, 19.0 and 19.5 and weak peaks at 7.5, 7.8, 11.7, 12.1, 15.1, 15.6, 16.5, 17.2, 17.6, 20.0, 20.8, 21.8, 22.7, 23.4, 24.5, 27.4 and 28.4.

When methanol is used as a co-solvent with toluene, ethylacetate or mixtures of toluene and ethylacetate, a crystalline BOC-protected 2R,3S—N-isobutyl-N-(2-hydroxy-3-amino-4-phenylbutyl)-p-nitrobenzene-sulfonylamide of Crystal Form I is obtained. The habit of Crystal Form II is prismatic (thick needle) and thus is amenable to easy filtration. Scanning Electron Microscopy (Hitachi S570 Scanning Electron Microscope) shows ‘fluted’ rod shaped particles that are up to ˜200.0 micron long. When measured by X-ray diffraction (Siemens D5000 X-Ray Diffractometer, 2θ, CuKα radiation) Crystal Form II shows strong peaks, at 5.3 and 8.3, moderate peaks at 6.7, 10.7, 13.4, 18.8, 19.6, 21.3 and 23.8 and weak peaks at 7.9, 17.3, 20.6 and 21.9.

By contrast, when no methanol is present and only ethyl acetate is used as the sole solvent, Crystal Form III is obtained and when no methanol is present and only toluene is used as the sole solvent, Crystal Form IV is obtained. BOC-protected 2R,3S—N-isobutyl-N-(2-hydroxy-3-amino-4-phenylbutyl)-p-nitrobenzene-sulfonylamide isolated as Crystal Form III or IV is difficult to filter as the crystal habit of both forms is that of fibrous needles which weave into a tight compact mass. From the low magnification micrographs (Philips XL30 SFEG Scanning Electron Microscope) large clusters of irregular shape are observed, there are also some very large aggregates. The high magnification micrographs show a variety of shapes from a fibrous network to very small lath shaped particles and some amorphous like clusters. When measured by X-ray diffraction (Siemens D5000 X-Ray Diffractometer, 2θ, CuKα radiation) Crystal Form III shows moderate peaks at 7.0 and 14.0 and weak peaks at 3.5, 8.7, 9.4, 10.1, 10.5, 11.1, 16.5, 17.6, 18.1, 19.6, 21.1 and 25.7, and Crystal Form IV shows a strong peak at 6.3, a moderate peak at 15.8 and weak peaks at 8.7, 9.5, 9.9 and 16.5.

In addition, when dichloromethane is used as solvent, Crystal Form V is obtained. When measured by X-ray diffraction (Siemens D5000 X-Ray Diffractometer, 2θ, CuKα radiation) Crystal Form V shows a strong peak at 6.8, a moderate peak at 13.6 and broad weak peaks at 3.6, 9.2, 10.7 and 19.5. Although the use of chlorinated solvents may not be advantageous for environmental or other reasons, the crystal forms obtainable by processes employing chlorinated solvents show advantage in ease of filtration as the crystal habit is prismatic (thick needle).

From thermogravimetric analysis of the aforementioned crystals forms, there is evidence that suggests that Crystal Forms I, II and V are solvates. Subjecting Crystal Forms I, II and V to heating, particularly under vacuum, appears to release solvent trapped in the crystal resulting in a new crystal structure being isolated. For example, Crystal Form II on heating becomes Crystal Form VI. When measured by X-ray diffraction (Siemens D5000 X-Ray Diffractometer, 2θ, CuKα radiation) Crystal Form VI shows moderate peaks at 6.8 and 8.8 and broad weak peaks at 5.6, 8.1, 14.3, 17.6 and 19.0.

In addition to methanol, other lower aliphatic alcohols were investigated. The solubility of BOC-protected 2R,3S—N-isobutyl-N-(2-hydroxy-3-amino-4-phenylbutyl)-p-nitrobenzene-sulfonylamide in propan-1-ol, propan-2-ol or butan-1-ol is too low to offer an economically viable process. For example, the solubility of BOC-protected 2R,3S—N-isobutyl-N-(2-hydroxy-3-amino-4-phenylbutyl)-p-nitrobenzene-sulfonylamide in propan-1-ol is less than 3% w/w at 60° C. The solubility of BOC-protected 2R,3S—N-isobutyl-N-(2-hydroxy-3-amino-4-phenylbutyl)-p-nitrobenzene-sulfonylamide in ethanol is better than that in C₃ and C₄-alcohols, but is still lower than the solubility in methanol. Crystals obtained from ethanol are thinner and smaller needle crystals (acicular as opposed to prismatic) and therefore would prove inferior to methanol from a processing perspective. Similarly, acetone and 2-pentanone yield small acicular crystals.

The BOC-protected 2R,3S—N-isobutyl-N-(2-hydroxy-3-amino-4-phenylbutyl)-p-nitrobenzene-sulfonylamide employed in the first and further aspects of the present invention may be obtained by any method known in the art. Preferably the BOC-protected 2R,3S—N-isobutyl-N-(2-hydroxy-3-amino-4-phenylbutyl)-p-nitrobenzene-sulfonylamide is prepared from a BOC-protected epoxide of Formula (1) or a halomethylalcohol of Formula (2).

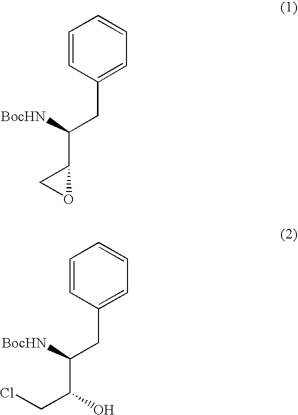

Suitable methods for the preparation of BOC-protected 2R,3S—N-isobutyl-N-(2-hydroxy-3-amino-4-phenylbutyl)-p-nitrobenzene-sulfonylamide are described in U.S. Pat. No. 5,585,397, U.S. Pat. No. 5,723,490, U.S. Pat. No. 5,783,701, WO94/05639, WO99/48885, WO01/046120, U.S. Pat. No. 6,548,706 and U.S. Pat. No. 6,852,887 the teachings of all of which are incorporated herein by reference.

The invention will now be described, without limitation, by the following examples.

### EXPERIMENTAL

### Example 1

Preparation of BOC-protected 2R,3S—N-isobutyl-N-(2-hydroxy-3-amino-4-phenylbutyl)-p-nitrobenzene-sulfonylamide.

BOC protected 2R,3S—N-isobutyl-N-(2-hydroxy-3-amino-4-phenylbutyl)amine (51.1 g, 0.152 mol) in toluene (12.5 eq, 175 g) was heated to 75° C. and aqueous potassium carbonate solution added (2 eq, 14.5% w/w, 291 g). With vigorous stirring, a solution of 4-nitrobenzenesulfonyl chloride (1.3 eq, 43.8 g) in ethyl acetate (9.7 eq, 130 g) was added. The solution was held at 75° C. for one hour before removing the aqueous phase. The organic phase was washed with hot water at 75° C. (3×200 g). Methanol was charged (205.7 g, 42.2 eq) and the mixture cooled to 43° C. over an hour. The mixture was then seeded with BOC-protected 2R,3S—N-isobutyl-N-(2-hydroxy-3-amino-4-phenylbutyl)-p-nitrobenzene-sulfonylamide of Crystal Form II (0.1 g) and then cooled at a rate of 3.5° C./h for 2 hrs, then at 5° C./hr for 2 hrs and then finally at 10° C./hr until 0° C. was reached. The solid was then isolated by filtration and washed with 10% v/v ethyl acetate/methanol (3×144 g).

The isolated crystals are prismatic (thick needle) and thus are amenable to easy filtration. Scanning Electron Microscopy (Hitachi S570 Scanning Electron Microscope) shows ‘fluted’ rod shaped particles that are up to ˜200.0 micron long. XRD analysis (Siemens D5000 X-Ray Diffractometer, 2θ, CuKα radiation) shows the crystals to be Crystal Form II with strong peaks at 5.3 and 8.3, moderate peaks at 6.7, 10.7, 13.4, 18.8, 19.6, 21.3 and 23.8 and Weak peaks at 7.9, 17.3, 20.6 and 21.9.

The product was dried in a vacuum oven at 50° C. Isolated yield=60.4 g (76%).

### Example 2

### General Method

Preparation of the different crystalline forms of BOC-protected 2R,3S—N-isobutyl-N-(2-hydroxy-3-amino-4-phenylbutyl)-p-nitrobenzene-sulfonylamide by equilibration in solvent.

BOC-protected 2R,3S—N-isobutyl-N-(2-hydroxy-3-amino-4-phenylbutyl)-p-nitrobenzene-sulfonylamide (0.5 g) is suspended in solvent (10 g). The suspension is raised to an elevated temperatures (typically 35-40° C.) for several days to allow transformation to the most stable form to take place, followed by slow cooling to room temperature. The solid was isolated by filtration and analysed by XRD and SEM.

### Example 2A

The general method was followed using methanol as solvent. The solid isolated was characterised by XRD (Type I)

### Example 2B

The general method was followed using a toluene, ethyl acetate and methanol mixture (39/23/38% w/w) as solvent. The solid isolated was characterised by XRD (Type II)

### Comparative Example 2C

The general method was followed using ethyl acetate as solvent. The solid isolated was characterised by XRD (Type III)

### Comparative Example 2D

The general method was followed using toluene as solvent. The solid isolated was characterised by XRD (Type IV)

### Example 2E

The general method was followed using dichloromethane as solvent. The solid isolated was characterised by XRD (Type V)

1. A process for preparing a BOC-protected 2R,3S—N-isobutyl-N-(2-hydroxy-3-amino-4-phenylbutyl)-p-nitrobenzene-sulfonylamide wherein the BOC-protected 2R,3S—N-isobutyl-N-(2-hydroxy-3-amino-4-phenylbutyl)-p-nitrobenzene-sulfonylamide is isolated from a solution comprising methanol and BOC-protected 2R,3S—N-isobutyl-N-(2-hydroxy-3-amino-4-phenylbutyl)-p-nitrobenzene-sulfonylamide.

2. A process according to claim 1 wherein the solution comprising methanol and BOC-protected 2R,3S—N-isobutyl-N-(2-hydroxy-3-amino-4-phenylbutyl)-p-nitrobenzene-sulfonylamide is a solution comprising BOC-protected 2R,3S—N-isobutyl-N-(2-hydroxy-3-amino-4-phenylbutyl)-p-nitrobenzene-sulfonylamide in methanol or a methanol containing solvent mixture.

3. A process according to claim 2 wherein the methanol containing solvent mixture is a methanol/toluene mixture, a methanol/ethyl acetate mixture or a methanol/toluene/ethyl acetate mixture.

4. A process according to claim 2, wherein when methanol containing solvent mixtures are employed, the methanol content is greater than 10%.

5. A process according to claim 2, wherein when tertiary mixtures of methanol containing solvent mixtures are employed, the other non-methanol solvents are preferably present in ratios ranging from 1:99 to 99:1, more preferably in ratios ranging from 25:75 to 75:25, and most preferably in ratios ranging from 60:40 to 40:60, for example 50:50.

6. A process according to claim 1 wherein the solution comprising methanol and BOC-protected 2R,3S—N-isobutyl-N-(2-hydroxy-3-amino-4-phenylbutyl)-p-nitrobenzene-sulfonylamide is obtained by dissolving BOC-protected 2R,3S—N-isobutyl-N-(2-hydroxy-3-amino-4-phenylbutyl)-p-nitrobenzene-sulfonylamide in methanol or methanol containing solvent mixtures, or by direct addition of methanol to a solution comprising BOC-protected 2R,3S—N-isobutyl-N-(2-hydroxy-3-amino-4-phenylbutyl)-p-nitrobenzene-sulfonylamide or by performing a solvent exchange on a solution comprising BOC-protected 2R,3S—N-isobutyl-N-(2-hydroxy-3-amino-4-phenylbutyl)-p-nitrobenzene-sulfonylamide whereby the solution solvent is exchanged for methanol or a methanol containing solvent mixture.

7. A process according to claim 1 wherein
(a) BOC-protected 2R,3S—N-isobutyl-N-(2-hydroxy-3-amino-4-phenylbutyl)-p-nitrobenzene-sulfonylamide is dissolved by heating in methanol or a methanol comprising solvent mixture to form a saturated or partially saturated methanol containing solution;
(b) optionally the saturated or partially saturated methanol containing solution is filtered to remove any insoluble material; then
(c) the saturated or partially saturated methanol containing solution is cooled to precipitate the BOC-protected 2R,3S—N-isobutyl-N-(2-hydroxy-3-amino-4-phenylbutyl)-p-nitrobenzene-sulfonylamide.

8. A process according to claim 1 wherein
(a) methanol is added to a heated saturated or partially saturated solution of BOC-protected 2R,3S—N-isobutyl-N-(2-hydroxy-3-amino-4-phenylbutyl)-p-nitrobenzene-sulfonylamide in a non-methanol solvent or solvent mixture;
(b) optionally the saturated or partially saturated methanol containing solution is filtered to remove any insoluble material; then
(c) the saturated or partially saturated methanol containing solution is cooled to precipitate the BOC-protected 2R,3S—N-isobutyl-N-(2-hydroxy-3-amino-4-phenylbutyl)-p-nitrobenzene-sulfonylamide.

9. A process according to claim 7 wherein the saturated or partially saturated methanol containing solution is seeded during the cooling in step (c).

10. A crystalline BOC-protected 2R,3S—N-isobutyl-N-(2-hydroxy-3-amino-4-phenylbutyl)-p-nitrobenzene-sulfonylamide obtainable by the processes according to claim 1.

11. A crystalline BOC-protected 2R,3S—N-isobutyl-N-(2-hydroxy-3-amino-4-phenylbutyl)-p-nitrobenzene-sulfonylamide of Crystal Form I which shows strong peaks at 3.9 and 8.2.

12. A crystalline BOC-protected 2R,3S—N-isobutyl-N-(2-hydroxy-3-amino-4-phenylbutyl)-p-nitrobenzene-sulfonylamide according to claim 11 which additionally shows moderate peaks at 9.3, 13.7 18.7, 19.0 and 19.5.

13. A crystalline BOC-protected 2R,3S—N-isobutyl-N-(2-hydroxy-3-amino-4-phenylbutyl)-p-nitrobenzene-sulfonylamide according to claim 12 which additionally shows weak peaks at 7.5, 7.8, 11.7, 12.1, 15.1, 15.6, 16.5, 17.2, 17.6, 20.0, 20.8, 21.8, 22.7, 23.4, 24.5, 27.4 and 28.4.

14. A crystalline BOC-protected 2R,3S—N-isobutyl-N-(2-hydroxy-3-amino-4-phenylbutyl)-p-nitrobenzene-sulfonylamide of Crystal Form II which shows strong peaks at 5.3 and 8.3.

15. A crystalline BOC-protected 2R,3S—N-isobutyl-N-(2-hydroxy-3-amino-4-phenylbutyl)-p-nitrobenzene-sulfonylamide according to claim 14 which additionally shows moderate peaks at 6.7, 10.7, 13.4, 18.8, 19.6, 21.3 and 23.8.

16. A crystalline BOC-protected 2R,3S—N-isobutyl-N-(2-hydroxy-3-amino-4-phenylbutyl)-p-nitrobenzene-sulfonylamide according to claim 15 which additionally shows and weak peaks at 7.9, 17.3, 20.6 and 21.9.

17. A crystalline BOC-protected 2R,3S—N-isobutyl-N-(2-hydroxy-3-amino-4-phenylbutyl)-p-nitrobenzene-sulfonylamide of Crystal Form VI which shows moderate peaks at 6.8 and 8.8 and preferably shows additional broad weak peaks at 5.6, 8.1, 14.3, 17.6 and 19.0.

18. A crystalline BOC-protected 2R,3S—N-isobutyl-N-(2-hydroxy-3-amino-4-phenylbutyl)-p-nitrobenzene-sulfonylamide of Crystal Form V which shows a strong peak at 6.8, and preferably shows additional moderate peak at 13.6 and broad weak peaks at 3.6, 9.2, 10.7 and 19.5.

19. A process according to claim 8 wherein the saturated or partially saturated methanol containing solution is seeded during the cooling in step (c).

20. A process according to claim 8 wherein the non-methanol solvent is toluene, ethyl acetate or toluene/ethyl acetate mixture.
